(19) United States
(12) Patent Application Publication
(10) Pub. No.: US 2020/0129513 A1
(43) Pub. Date: Apr. 30, 2020

(54) COMPOSITIONS AND METHODS FOR TREATING HEMATOLOGIC MALIGNANCIES

(71) Applicant: UNIVERSITY OF ROCHESTER, ROCHESTER, NY (US)

(72) Inventors: Charles Chiyuan Chu, Pittsford, NY (US); Omar Salah Aljitawi, Pittsford, NY (US)

(21) Appl. No.: 16/668,748

(22) Filed: Oct. 30, 2019

Related U.S. Application Data
(60) Provisional application No. 62/753,262, filed on Oct. 31, 2018.

(51) Int. Cl. A61K 31/517; C07K 16/28; A61P 35/02

(52) U.S. Cl. CPC A61K 31/517; C07K 2317/76; A61P 35/02; C07K 16/2863

(57) ABSTRACT

The invention provides novel methods and pharmaceutical compositions for treating various hematologic malignancies and related diseases and conditions.

### PRIORITY CLAIMS AND RELATED APPLICATIONS

This application claims the benefit of priority to U.S. Provisional Application No. 62/753,262, filed Oct. 31, 2018, the entire content of which is incorporated herein by reference for all purposes.

### TECHNICAL FIELD OF THE INVENTION

The invention generally relates to therapeutics and treatment methods for certain diseases and conditions. More particularly, the invention provides novel methods and compositions for treating various hematologic malignancies and related diseases and conditions.

### BACKGROUND OF THE INVENTION

Hematologic malignancies are forms of cancer that begin in the cells of blood-forming tissue, for example, the bone marrow or in the cells of the immune system. Examples of hematologic cancers include acute and chronic leukemia, lymphomas, multiple myeloma and myelodysplastic syndromes.

Leukemia, a type of cancer that occurs in the bone marrow and in the blood, is generally classified into two types: lymphocytic leukemia that involves lymphocytes and myelogenous leukemia involves granulocytes. Lymphoid leukemias include predominantly acute lymphoblastic or lymphocytic leukemia (ALL) and chronic lymphocytic leukemia (CLL). Myeloid leukemias include predominantly acute myeloblastic leukemia (AML) and chronic myelogenous leukemia (CIVIL). Additional myeloid malignancies include myelodysplastic syndrome, essential thrombocythemia, polycythemia vera and myelofibrosis. Lymphoma, a type of lymphoid cancer that develops in the lymphatic system, also falls into two general types of lymphoma, i.e., Hodgkin lymphoma and non-Hodgkin lymphoma (NHL). In Hodgkin lymphoma, the cancer spreads from one group of lymph nodes to another in a certain order. In NHL, the cancer spreads from one group of lymph nodes to another in a random order. There are many types of NHL, including Burkitt lymphoma (BL), follicular lymphoma (FL), diffuse large B-cell lymphoma (DLBCL) and mantle cell lymphma (MCL). Myeloma is a lymphoid cancer that causes the antibody-producing plasma cells to form a tumor in the bone marrow. Myeloma is usually found in multiple places in the body, thus often called multiple myeloma.

Hematologic malignancies place significant burdens on the healthcare system and the society in general. For examples, around 174,170 new cases of lymphoid and myeloid malignances occurred in the U.S. alone in 2018. About 10% of all new cancers diagnosed in 2018 in the U.S. are hematologic malignancies, which rank as the 6th leading cause of cancer-related death.

Existing treatments are often inadequate in terms of clinical effectiveness. At the same time, available therapeutics often lead to undesirable side effects. Treatment of hematologic malignancies usually includes “watchful waiting” or symptomatic treatment for indolent malignancies. More aggressive forms of treatment that includes chemotherapy, radiotherapy, immunotherapy or a bone marrow transplant.

Recent advances in cancer therapeutics are transforming treatment of some hematological malignancies and have given new hope to patients and health care staff about the possibility of cures. For example, treatment of CLL by small molecule inhibitors of Bruton's tyrosine kinase (BTK) has provided encouraging patient responses. However, BTK inhibitor treatment has not proven curative and relapses are often very aggressive with poor patient outcome. In other hematological malignancies, such as AML, the successful use of small molecule inhibitors has been very limited, in part due to the resistance of AML cancer “stem” cells residing in the tumor microenvironment. Therefore, further advances in drug targeting of hematological malignancies are needed.

There exists an urgent unmet need for novel therapeutics and treatment methods that provide improved clinical effectiveness with reduced side effects.

### SUMMARY OF THE INVENTION

The invention is based in part on the unexpected discovery that inhibitors of epidermal growth factor receptor (EGFR) family of receptors can be used to effectively treat one or more hematological malignancies, or related diseases and conditions.

As disclosed herein, in one aspect, the invention generally relates to a method for treating a hematologic malignancy, or a related disease or condition. The method includes administering to a subject in need thereof a pharmaceutical composition comprising an inhibitor of a member of the EGFR family, or a pharmaceutically acceptable salt, ester or pro-drug thereof.

In another aspect, the invention generally relates to a method for inhibiting or slowing tumor growth of a hematologic malignancy. The method includes administering to a subject in need thereof a pharmaceutical composition comprising an inhibitor of a member of epidermal growth factor receptor (EGFR) family, or a pharmaceutically acceptable salt, ester or pro-drug thereof.

In yet another aspect, the invention generally relates to a pharmaceutical composition comprising an inhibitor of a member of the EGFR family, or a pharmaceutically acceptable salt, ester or pro-drug thereof, in an amount effective in the treatment of a hematologic malignancy, or a related disease or condition, in a mammal, including a human, and a pharmaceutically acceptable excipient, carrier, or diluent.

In yet another aspect, the invention generally relates to a pharmaceutical composition comprising an inhibitor of a member of the EGFR family, or a pharmaceutically acceptable salt, ester or pro-drug thereof, in an amount effective in inhibiting or slowing tumor growth of a hematologic malignancy, or a related disease or condition thereof, in a mammal, including a human, and a pharmaceutically acceptable excipient, carrier, or diluent.

In yet another aspect, the invention generally relates to a unit dosage form that comprises a compound or a pharmaceutical composition disclosed herein.

In yet another aspect, the invention generally relates to a method for affecting a microenvironmental factor of a hematological cancer cell. The method includes administering to a subject suffering from a hematologic malignancy, or a related disease or condition, an inhibitor of a member of the EGFR family, or a pharmaceutically acceptable salt, ester or pro-drug thereof.

The present invention may fundamentally alter the treatment protocol for treatment of hematologic malignancies and related disease and conditions by providing a novel therapeutic target aimed at the microenvironmental of hematological cancer cells.

### BRIEF DESCRIPTION OF THE DRAWINGS

FIG. 1. Afatinib (AFA) reduces CLL cell viability in a dose dependent manner (IC50 0.4 μM) as measured by Cell Titer-Blue Cell Viability Assay.

FIG. 2. Afatinib (AFA) reduces CLL cell viability in a dose dependent manner (IC50 0.3 μM) as measured by Cell Titer-Glo 2.0 Assay.

FIG. 3. Afatinib (AFA) induces CLL cell death in a dose dependent manner (IC50 12 μM).

FIG. 4. Afatinib (AFA) reduces CLL cell viability (IC50 0.5 μM) at a lower concentration than ibrutinib (IBR, IC50 10 μM).

FIG. 5. Current AML Model. EGFR signaling pathway from extracellular matrix promotes adherent AML cells to trigger glycolysis and become chemoresistant.

FIG. 6. Afatinib (AFA) reduces cell viability in two different AML patient samples in a dose dependent manner (IC50 2.1 and 2.6 μM).

FIG. 7. Afatinib (AFA) induces AML cell death in a dose dependent manner (IC50 19 μM).

FIG. 8. Model of cancer cell and EGFR family interactions in the tumor microenvironment of hematological malignancies.

FIG. 9. Afatinib (AFA) reduces cell viability of a p53 mutated Non-Hodgkin Lymphoma (NHL) cell line in a dose dependent manner (3 μM), whereas dexamethasone (DEX) does not.

FIG. 10. Afatinib (AFA) induces cell death of a p53 mutated Non-Hodgkin Lymphoma (NHL) cell line in a dose dependent manner (22 μM), whereas dexamethasone (DEX) does not.

FIG. 11. EGFR family of receptors are expressed in CLL cells.

FIG. 12. EGFR family of receptors are highly expressed in AML cells.

FIG. 13. EGFR family of receptors are highly expressed in BL cells.

FIG. 14. EGFR family of receptors are expressed in FL cells.

FIG. 15. EGFR family of receptors are expressed in DLBCL cells.

FIG. 16. EGFR family of receptors are expressed in MCL cells.

FIG. 17. EGFR family of receptors are highly expressed in myeloma cells.

FIG. 18. EGFR family of receptors are highly expressed in ALL cells.

FIG. 19. EGFR family ligands are expressed in BL tissue biopsies.

FIG. 20. EGFR family ligands are expressed in FL tissue biopsies.

FIG. 21. EGFR family ligands are expressed in DLBCL tissue biopsies.

FIG. 22. EGFR family ligands are expressed in MCL tissue biopsies.

FIG. 23. NRG1 protein expression in a representative NHL tissue biopsy.

FIG. 24. NRG2 protein expression in a representative NHL tissue biopsy.

FIG. 25. NRG4 protein expression in a representative NHL tissue biopsy.

FIG. 26. AREG protein expression in a representative NHL tissue biopsy.

FIG. 27. HBEGF protein expression in a representative NHL tissue biopsy.

FIG. 28. TGFA protein expression in a representative NHL tissue biopsy.

FIG. 29. HER3 protein expression in a representative NHL (Non-Hodgkin Lymphoma) tissue biopsy.

FIG. 30. HER3 protein expression in a representative CLL (chronic lymphocytic leukemia) lymph node (LN) biopsy.

FIG. 31. Full length HER3 protein is detectable in AML (acute myeloid leukemia) primary cell extracts, either from suspension cells grown in tissue culture media or from suspension cells isolated from a tissue culture matrix or from adherent cells isolated from a tissue culture matrix.

FIG. 32. Full length HER3 protein is detectable in CLL (chronic lymphocytic leukemia) primary cells, as suspension cells grown in serum-free tissue culture media (AimV).

FIG. 33. Shorter than full-length HER4 protein is detectable in AML (n=4) and CLL (n=4) primary cells, as suspension cells prepared from frozen aliquots. The size is similar to that expected for just the HER4 intracellular domain (ICD) fragment.

FIG. 34. NRG1 protein staining is moderate to strong cytoplasmic staining in most lymphomas, but negative in remaining cancer cell types.

FIG. 35. NRG1 protein expression in a representative CLL (chronic lymphocytic leukemia) lymph node (LN) biopsy.

FIG. 36. Shorter than full-length NRG1 protein is detectable in AML (n=4) and CLL (n=4) primary cells, as suspension cells prepared from frozen aliquots. The size is similar to that expected for the NRG1 extracellular domain (ECD) fragment.

### DEFINITIONS

Unless defined otherwise, all technical and scientific terms used herein have the same meaning as commonly understood by one of ordinary skill in the art to which this invention belongs. General principles of organic chemistry, as well as specific functional moieties and reactivity, are described in “Organic Chemistry”, Thomas Sorrell, University Science Books, Sausalito: 2006.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic methods well known in the art, and subsequent recovery of the pure enantiomers.

As used herein, the term “administering” refers to oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Suitable routes of administration for a particular patient will depend on the nature and severity of the disease or condition being treated or the nature of the therapy being used and on the nature of the active compound.

Administration may be by any suitable route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

By “co-administer” it is meant that a composition described herein is administered at the same time, just prior to, or just after the administration of one or more additional therapies (e.g., anti-cancer agent or chemotherapeutic).

The compound of the invention can be administered alone or can be co-administered to the patient. Co-administration is meant to include simultaneous or sequential administration of the compound individually or in combination (more than one compound or agent). Thus, the preparations can also be combined, when desired, with other active substances (e.g., to reduce metabolic degradation).

As used herein, the terms “disease,” “condition,” and “disorder” are used interchangeably herein and refer to a state of being or health status of a patient or subject capable of being treated with a compound, pharmaceutical composition, or method provided herein.

As used herein, the term “effective amount” of an active agent refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the patient.

As used herein, the terms “inhibition,” “inhibit” and “inhibiting” and the like in reference to a biological target (e.g., EGFR) inhibitor interaction refers to negatively affecting (e.g., decreasing) the activity or function of the protein relative to the activity or function of the protein in the absence of the inhibitor. In embodiments, inhibition means negatively affecting (e.g. decreasing) the concentration or levels of the protein relative to the concentration or level of the protein in the absence of the inhibitor. In embodiments, inhibition refers to reduction of a disease or symptoms of disease. In embodiments, inhibition refers to a reduction in the activity of a particular protein target. Inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g., an inhibitor binds to the target protein). In embodiments, inhibition refers to a reduction of activity of a target protein from an indirect interaction (e.g., an inhibitor binds to a protein that activates the target protein, thereby preventing target protein activation).

As used herein, the terms “isolated” or “purified” refer to a material that is substantially or essentially free from components that normally accompany it in its native state. Purity and homogeneity are typically determined using analytical chemistry techniques such as polyacrylamide gel electrophoresis or high-performance liquid chromatography.

As used herein, a “pharmaceutically acceptable form” of a disclosed compound includes, but is not limited to, pharmaceutically acceptable salts, esters, hydrates, solvates, isomers, prodrugs, and isotopically labeled derivatives thereof. In one embodiment, a “pharmaceutically acceptable form” includes, but is not limited to, pharmaceutically acceptable salts, esters, prodrugs and isotopically labeled derivatives thereof. In some embodiments, a “pharmaceutically acceptable form” includes, but is not limited to, pharmaceutically acceptable isomers and stereoisomers, prodrugs and isotopically labeled derivatives thereof.

In certain embodiments, the pharmaceutically acceptable form is a pharmaceutically acceptable salt. As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, organic acids from which salts can be derived include, for example, acetic acid, propionic acid, glycolic acid, pyruvic acid, oxalic acid, lactic acid, trifluoracetic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

The salts can be prepared in situ during the isolation and purification of the disclosed compounds, or separately, such as by reacting the free base or free acid of a parent compound with a suitable base or acid, respectively. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Organic bases from which salts can be derived include, for example, primary, secondary, and tertiary amines, substituted amines, including naturally occurring substituted amines, cyclic amines, basic ion exchange resins, and the like, such as isopropylamine, trimethylamine, diethylamine, triethylamine, tripropylamine, and ethanolamine. In some embodiments, the pharmaceutically acceptable base addition salt can be chosen from ammonium, potassium, sodium, calcium, and magnesium salts.

In certain embodiments, the pharmaceutically acceptable form is a “solvate” (e.g., a hydrate). As used herein, the term “solvate” refers to compounds that further include a stoichiometric or non-stoichiometric amount of solvent bound by non-covalent intermolecular forces. The solvate can be of a disclosed compound or a pharmaceutically acceptable salt thereof. Where the solvent is water, the solvate is a “hydrate.” Pharmaceutically acceptable solvates and hydrates are complexes that, for example, can include 1 to about 100, or 1 to about 10, or 1 to about 2, about 3 or about 4, solvent or water molecules. It will be understood that the term “compound” as used herein encompasses the compound and solvates of the compound, as well as mixtures thereof.

In certain embodiments, the pharmaceutically acceptable form is a prodrug. As used herein, the term “prodrug” (or “pro-drug”) refers to compounds that are transformed in vivo to yield a disclosed compound or a pharmaceutically acceptable form of the compound. A prodrug can be inactive when administered to a subject, but is converted in vivo to an active compound, for example, by hydrolysis (e.g., hydrolysis in blood). In certain cases, a prodrug has improved physical and/or delivery properties over the parent compound. Prodrugs can increase the bioavailability of the compound when administered to a subject (e.g., by permitting enhanced absorption into the blood following oral administration) or which enhance delivery to a biological compartment of interest (e.g., the brain or lymphatic system) relative to the parent compound. Exemplary prodrugs include derivatives of a disclosed compound with enhanced aqueous solubility or active transport through the gut membrane, relative to the parent compound.

The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein.

Prodrug forms often offer advantages of solubility, tissue compatibility, or delayed release in the mammalian organism. (See, Bundgard, Design of Prodrugs, pp. 7-9,21-24, Elsevier, Amsterdam 1985 and Silverman, The Organic Chemistry of Drug Design and Drug Action, pp. 352-401, Academic Press, San Diego, Calif., 1992). Prodrugs commonly known in the art include well-known acid derivatives, such as, for example, esters prepared by reaction of the parent acids with a suitable alcohol, amides prepared by reaction of the parent acid compound with an amine, basic groups reacted to form an acylated base derivative, etc. Other prodrug derivatives may be combined with other features disclosed herein to enhance bioavailability. As such, those of skill in the art will appreciate that certain of the presently disclosed compounds having free amino, arnido, hydroxy or carboxylic groups can be converted into prodrugs. Prodrugs include compounds having a carbonate, carbamate, amide or alkyl ester moiety covalently bonded to any of the above substituents disclosed herein.

Exemplary advantages of a prodrug can include, but are not limited to, its physical properties, such as enhanced water solubility for parenteral administration at physiological pH compared to the parent compound, or it can enhance absorption from the digestive tract, or it can enhance drug stability for long-term storage.

As used herein, the term “pharmaceutically acceptable” excipient, carrier, or diluent refers to a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

As used herein, the term “prodrug” (or “pro-drug”) refers to a pharmacological derivative of a parent drug molecule that requires biotransformation, either spontaneous or enzymatic, within the organism to release the active drug. Such prodrugs are pharmaceutically active in vivo, when they undergo solvolysis under physiological conditions or undergo enzymatic degradation. Prodrug compounds herein may be called single, double, triple, etc., depending on the number of biotransformation steps required to release the active drug within the organism, and the number of functionalities present in a precursor-type form.

Prodrug forms often offer advantages of solubility, tissue compatibility, or delayed release in the mammalian organism. (See, Bundgard, Design of Prodrugs, pp. 7-9,21-24, Elsevier, Amsterdam 1985 and Silverman, The Organic Chemistry of Drug Design and Drug Action, pp. 352-401, Academic Press, San Diego, Calif., 1992). Prodrugs commonly known in the art include well-known acid derivatives, such as, for example, esters prepared by reaction of the parent acids with a suitable alcohol, amides prepared by reaction of the parent acid compound with an amine, basic groups reacted to form an acylated base derivative, etc. Of course, other prodrug derivatives may be combined with other features disclosed herein to enhance bioavailability. As such, those of skill in the art will appreciate that certain of the presently disclosed compounds having free amino, amido, hydroxy or carboxylic groups can be converted into prodrugs. Prodrugs include compounds having an amino acid residue, or a polypeptide chain of two or more (e.g., two, three or four) amino acid residues which are covalently joined through peptide bonds to free amino, hydroxy or carboxylic acid groups of the presently disclosed compounds. The amino acid residues include the 20 naturally occurring amino acids commonly designated by three letter symbols and also include 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Prodrugs also include compounds having a carbonate, carbamate, amide or alkyl ester moiety covalently bonded to any of the above substituents disclosed herein.

As used herein, the term “subject” refers to any animal (e.g., a mammal), including, but not limited to humans, non-human primates, rodents, and the like, which is to be the recipient of a particular treatment. A subject to which administration is contemplated includes, but is not limited to, humans (e.g., a male or female of any age group, e.g., a pediatric subject (e.g., infant, child, adolescent) or adult subject (e.g., young adult, middle-aged adult or senior adult)) and/or other non-human animals, for example, non-human mammals (e.g., primates (e.g., cynomolgus monkeys, rhesus monkeys); commercially relevant mammals such as cattle, pigs, horses, sheep, goats, cats, and/or dogs), rodents (e.g., rats and/or mice), etc. In certain embodiments, the non-human animal is a mammal. The non-human animal may be a male or female at any stage of development. A non-human animal may be a transgenic animal. Typically, the terms “subject” and “patient” are used interchangeably herein in reference to a human subject.

As used herein, the terms “treatment” or “treating” a disease or disorder refers to a method of reducing, delaying or ameliorating such a condition before or after it has occurred. Treatment may be directed at one or more effects or symptoms of a disease and/or the underlying pathology. The treatment can be any reduction and can be, but is not limited to, the complete ablation of the disease or the symptoms of the disease. Treating or treatment thus refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters, for example, the results of a physical examination, neuropsychiatric exams, and/or a psychiatric evaluation. As compared with an equivalent untreated control, such reduction or degree of amelioration may be at least 5%, 10%, 20%, 40%, 50%, 60%, 80%, 90%, 95%, or 100% as measured by any standard technique.

Treatment methods include administering to a subject a therapeutically effective amount of a compound described herein. The administering step may be a single administration or may include a series of administrations. The length of the treatment period depends on a variety of factors, such as the severity of the condition, the patient's age, the concentration of the compound, the activity of the compositions used in the treatment, or a combination thereof. It will also be appreciated that the effective dosage of an agent used for the treatment may increase or decrease over the course of a particular treatment regime. Changes in dosage may result and become apparent by standard diagnostic assays known in the art. In some instances, chronic administration may be required. For example, the compositions are administered to the subject in an amount and for a duration sufficient to treat the patient.

As used herein, the term “low dosage” refers to at least 5% less (e.g., at least 10%, 20%, 50%, 80%, 90%, or even 95%) than the lowest standard recommended dosage of a particular compound formulated for a given route of administration for treatment of any human disease or condition. For example, a low dosage of an agent that is formulated for administration by inhalation will differ from a low dosage of the same agent formulated for oral administration.

As used herein, the term “high dosage” is meant at least 5% (e.g., at least 10%, 20%, 50%, 100%, 200%, or even 300%) more than the highest standard recommended dosage of a particular compound for treatment of any human disease or condition.

### DETAILED DESCRIPTION OF THE INVENTION

The invention provides a unique approach to treatment of hematologic malignancies and related diseases and conditions. The therapeutic methods and compositions provided herein can benefit cancer patients suffering from AML, CLL and other hematologic malignancies with improved treatment outcome and an increased survival rate.

The unique approach disclosed herein is based on a novel target for hematological malignancies. As first disclosed by the present inventors, the EGFR family of receptors is critical to the survival of hematological cancer cells, especially the cancer “stem” cells, in their tumor microenvironment. In particular, signaling through the EGFR family of receptors is vital to the sustained viability of cancer cells in hematological malignancies, both lymphoid and myeloid.

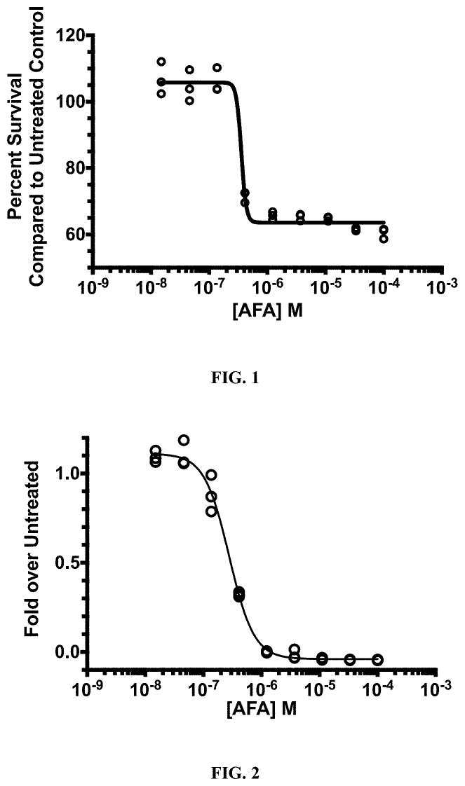

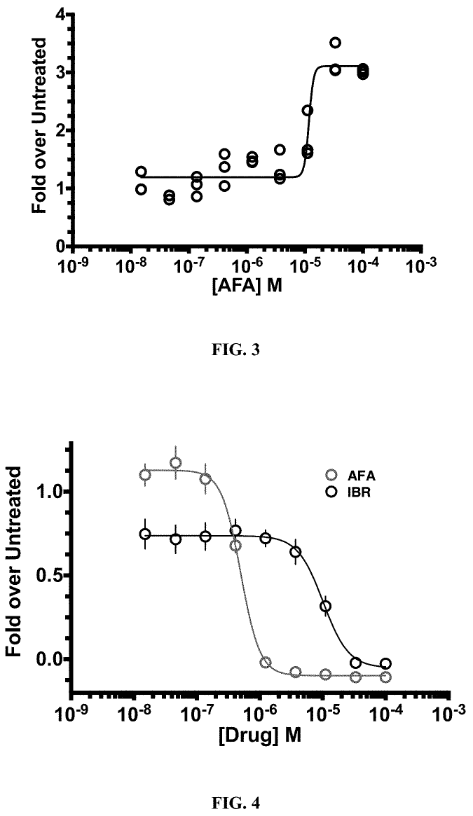

Among lymphoid malignancies, BTK inhibitors have been used to treat CLL. Relatively high doses of BTK inhibitors are needed to reduce CLL cell viability in vitro, suggesting a common off-target effect of these inhibitors may be responsible. The common off-target with the best binding among effective BTK inhibitors was ERRB2 in the EGFR family. Initial studies with afatinib (an EGFR family inhibitor) showed that an EGFR family inhibitor can reduce the viability of CLL cells (FIGS. 1-2) and increase CLL cell death (FIG. 3). This reduction of CLL cell viability by this EGFR family inhibitor is at a 20-fold lower concentration than ibrutinib, a strong BTK inhibitor (FIG. 4). These results support the use of EGFR family inhibitors (e.g., afatinib) in the treatment of lymphoid malignancies, such as CLL. Additionally, because a much lower dose of afatinib was needed than ibrutinib, EGFR family inhibitors could potentially be more effective and have less side effects than current BTK inhibitors in use to treat lymphoid malignancies such as CLL.

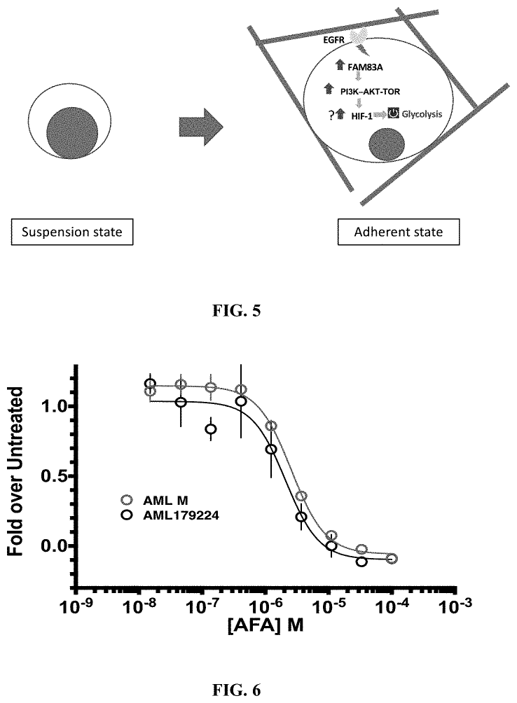

Among myeloid malignancies, AML is difficult to treat because the AML cancer “stem” cells are particularly insensitive to current therapies. Among the tumor, rare AML “stem” cells are able to self-renew and regenerate the entire range of AML cell phenotypes found in a tumor. Thus, therapies that target the AML “stem” cell are critically needed to provide a durable treatment response in AML. Additionally, cancer “stem” cells may be important in other hematological malignancies. Therefore, treatments that target cancer “stem” cells may be needed in other malignancies as well. Presently disclosed studies with an AML tumor microenvironment model indicate that EGFR signaling is vital to the survival of AML cancer stem cells as summarized in FIG. 5. Adherent human AML “stem” cells significantly upregulate FAM83A RNA expression, which promotes EGFR family signaling. This leads to PI3K-AKT-mTOR and MAPK signaling activation, resulting in the switch to glycolysis as the dominant energy production mechanism, a hallmark of cancer. This indicates that therapies that block EGFR family signaling may be effective in reducing survival of AML “stem” cells and possibly “stem” cells in other malignancies as well.

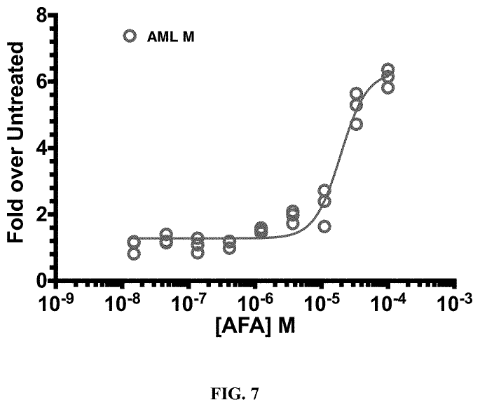

Leukemic cells are cancer cells that have moved out of the tumor microenvironment into the blood circulation. These circulating “suspension” cells may be especially sensitive to EGFR family signaling blockade, because these cells are no longer receiving maximal EGFR family signaling from the tumor microenvironment. Treatment of primary AML suspension cells with an EGFR family inhibitor (afatinib was chosen as the model drug) in vitro resulted in reduced AML cell viability (FIG. 6) and increased AML cell death (FIG. 7). These results and those from CLL (FIGS. 1-4) support the approach of EGFR family signaling inhibition to eliminate circulating leukemic cells. Furthermore, these results and the AML “stem” cell studies support the use of EGFR inhibitors, such as afatinib, in the treatment of myeloid malignancies.

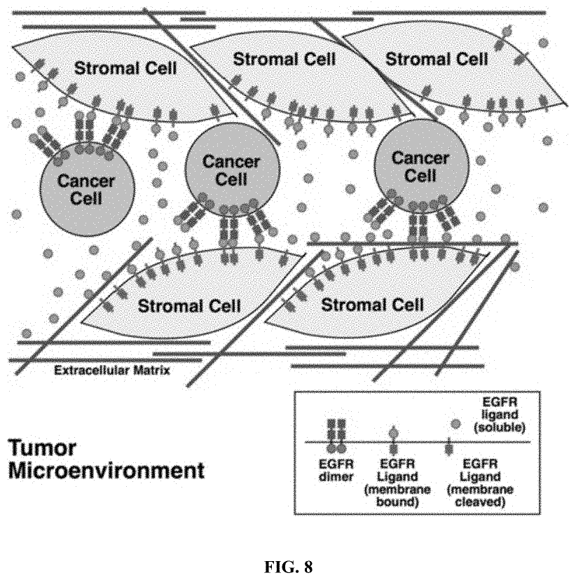

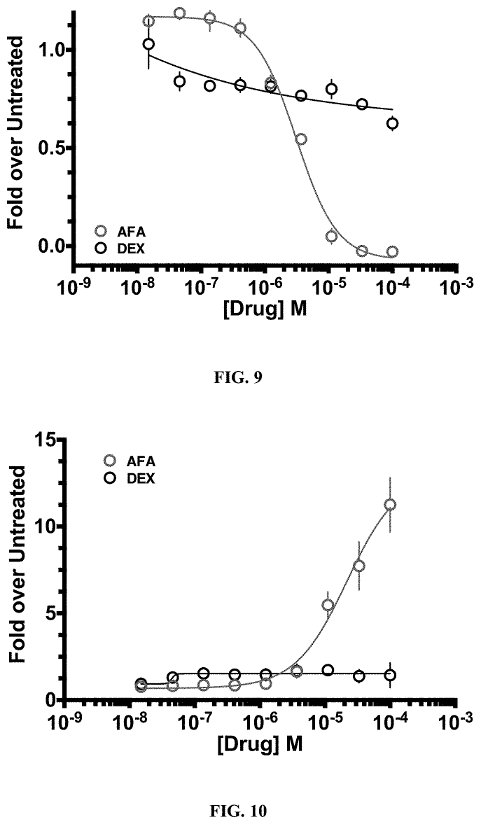

Based on our CLL and AML studies, the present inventors developed a general model of cancer cell and EGFR family (in particular, HER3 and HER4) interactions in the tumor microenvironment of hematological malignancies (FIG. 8). This model predicts a decrease in viability of many types of hematological malignancies after inhibition of EGFR family signaling. EGFR family inhibition (afatinib was chosen as the model drug) was tested on an NHL-derived cell line, CA46. This lymphoid cell line is derived from BL, a particularly aggressive type of NHL, Afatinib reduced NHL cell line viability (FIG. 9) and increased NHL cell line death (FIG. 10). These results support the use of EGFR inhibitors, such as afatinib, in the treatment of NHL and lymphoid malignancies.

Furthermore, the CA46 cell line is mutated in p53. Mutations in p53 are associated with more aggressive disease and are often difficult to treat, leading to poor patient outcomes. New therapies that target p53 mutated hematological malignancies are greatly needed. Afatinib decreases cell viability (FIG. 9) and increases cell death (FIG. 10) in this p53 mutated cell line. Therefore, EGFR family inhibition may be a therapeutic intervention that is not dependent on an intact p53 pathway and may be especially useful for patients with hematological malignancies containing p53 mutations.

Dexamethasone is part of current chemotherapeutic regimens for many lymphoid malignancies, including BL, and is cytotoxic towards these cancers. The efficacy of EGFR family inhibition was compared to dexamethasone treatment of the BL-derived cell line CA46. Afatinib, as the model EGFR family inhibitor, reduced cell viability (IC50 3 μM) and increased cell death (IC50 22 μM), whereas dexamethasone did not (FIGS. 9-10). Thus, EGFR family inhibition shows improved efficacy in treatment of hematological malignancies as compared to current chemotherapies.

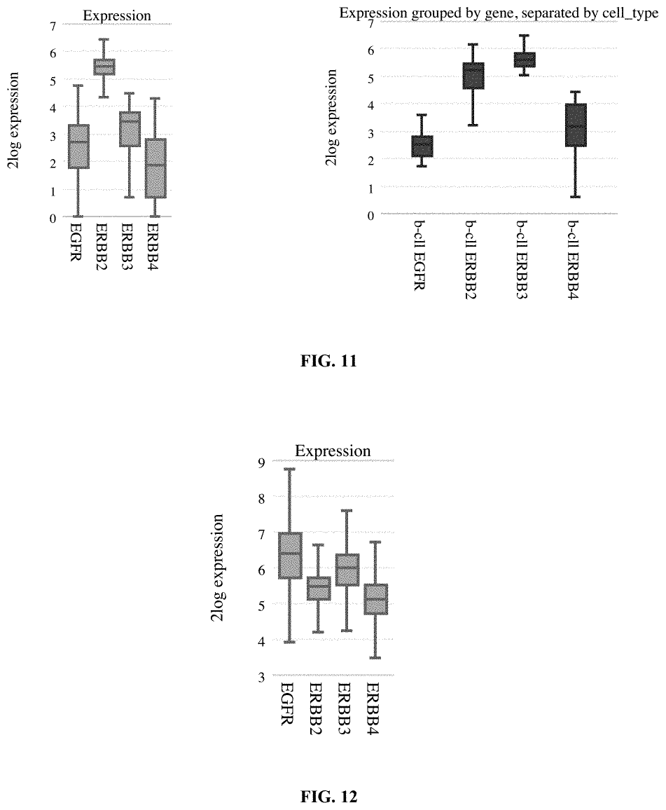

Gene expression of EGFR family members (EGFR, ERBB2, ERBB3, ERBB4) is found in all tested hematological malignancies: CLL, AML, BL, FL, DLBCL, MCL, myeloma, ALL (FIGS. 11-18). EGFR family RNA expression results support the therapeutic use of EGFR family inhibitors in these malignancies.

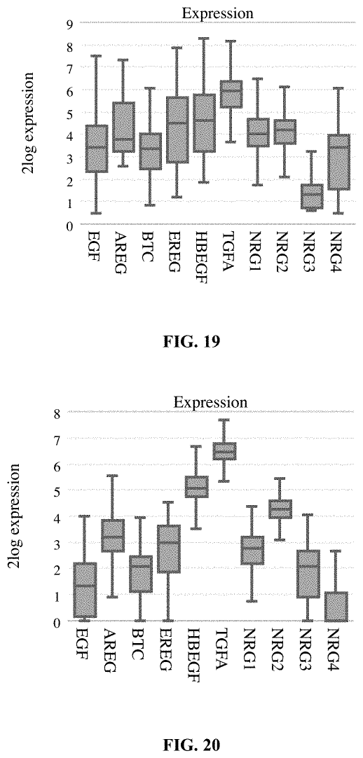

Gene expression of EGFR family ligands (EGF, AREG, BTC, EREG, HBEGF, TGFA, NRG1, NRG2, NRG3, NRG4, (except EPGN was not tested) is found in biopsies of all tested hematological malignancies: BL, FL, DLBCL, MCL (FIGS. 19-22). EGFR family ligands RNA expression results support the therapeutic use of EGFR family inhibitors in these malignancies.

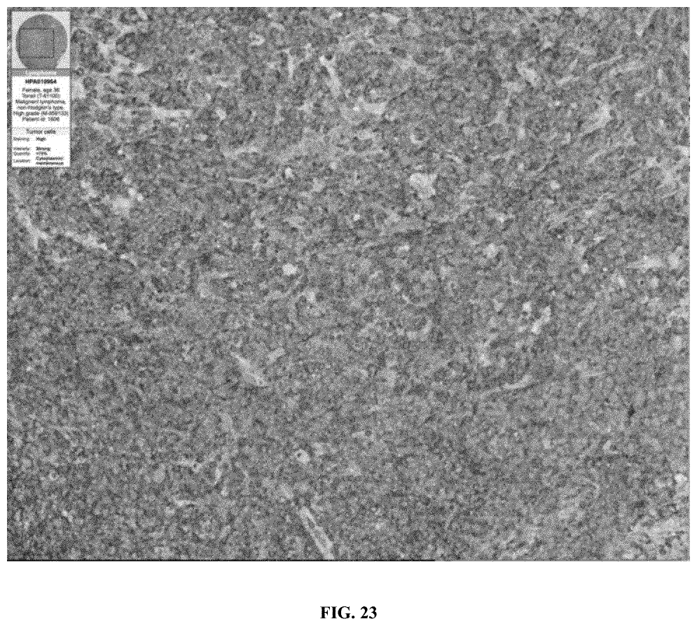

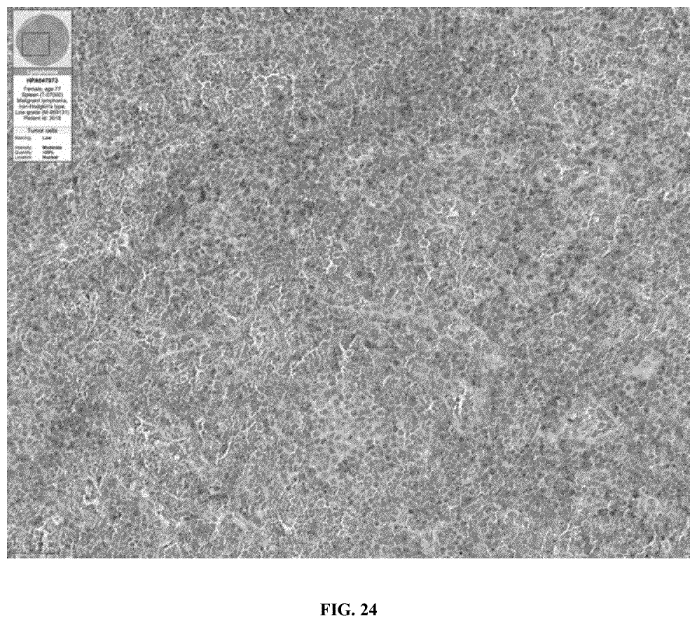

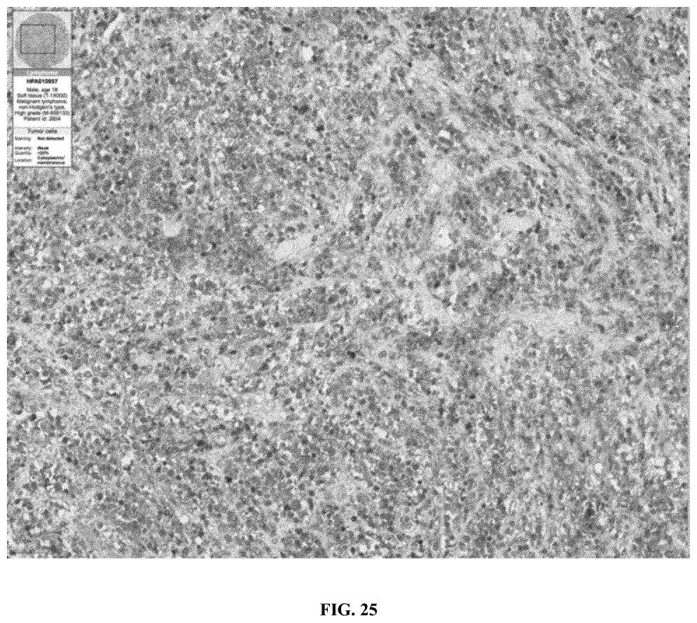

Protein expression of EGFR family ligands, where antibodies are available for immunohistochemistry staining (AREG, HBEGF, TGFA, NRG1, NRG2, NRG4), is found in NHL (FIGS. 23-28). EGFR family ligands protein expression results support the therapeutic use of EGFR family inhibitors in these malignancies.

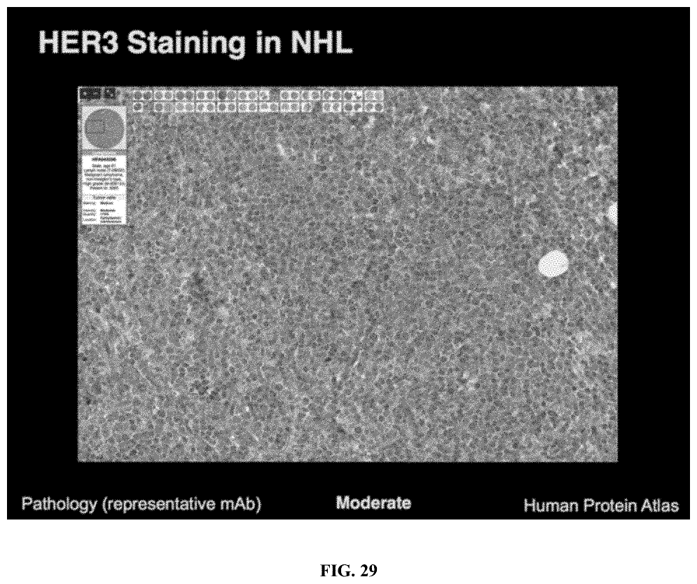

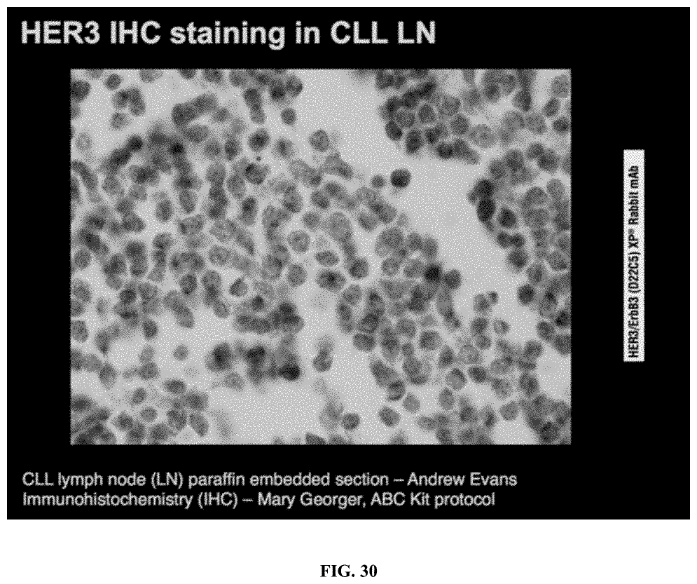

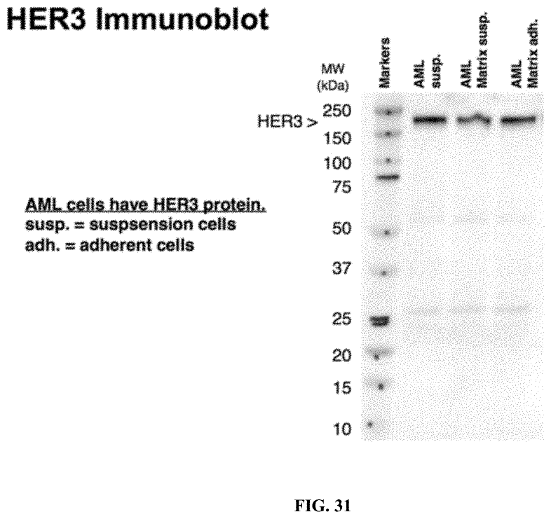

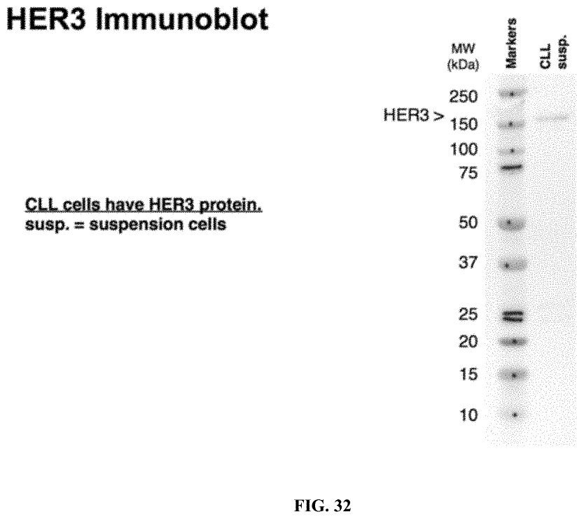

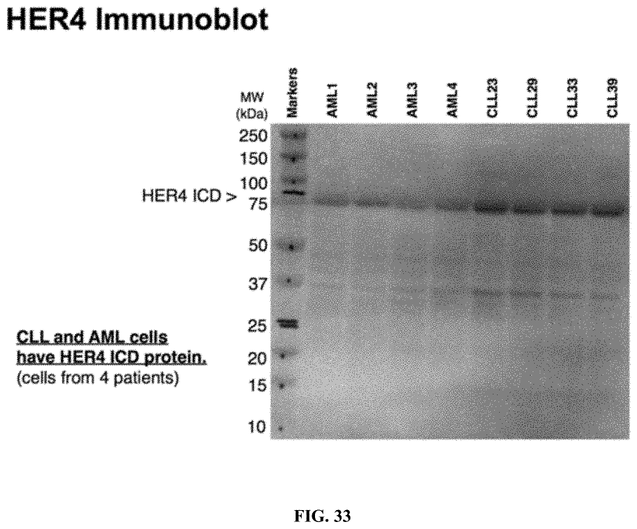

It was noted that in the EGFR family members responsible for maintaining the viability of hematological malignant cells, 75% of NHL tissue biopsies were positive for HER3 by IHC in the Human Protein Atlas (FIG. 29). In experiments disclosed herein, HER3 is detectable by IHC in CLL lymph node biopsies (FIG. 30). Furthermore, full-length HER3 protein was detectable by immunoblot in protein extracts from primary AML cells, including AML cells cultured in a matrix (Decellularized Wharton's Jelly Matrix (DWJM)) designed to simulate the tumor microenvironment (FIG. 31). Similarly, immunoblots of protein extracts from primary CLL cells have detectable full length HER3 protein (FIG. 32). HER4 protein was also seen in primary AML and CLL cell protein extracts, although in this case a truncated HER4 consistent with the intracellular domain (ICD) fragment was detected (FIG. 33).

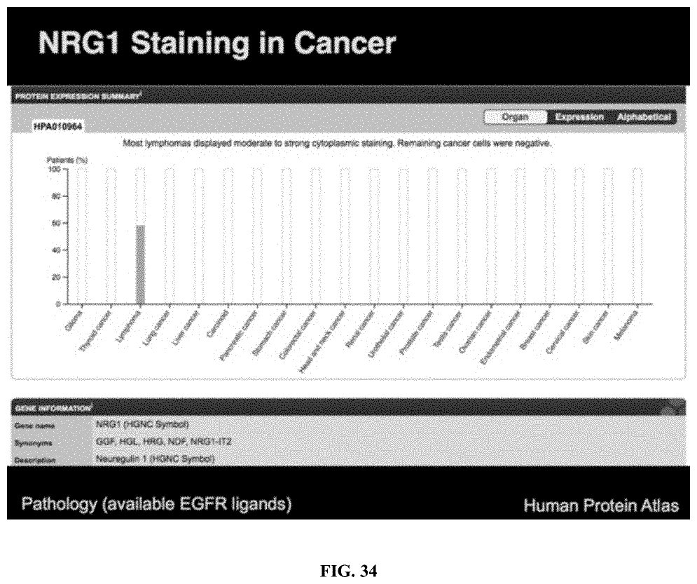

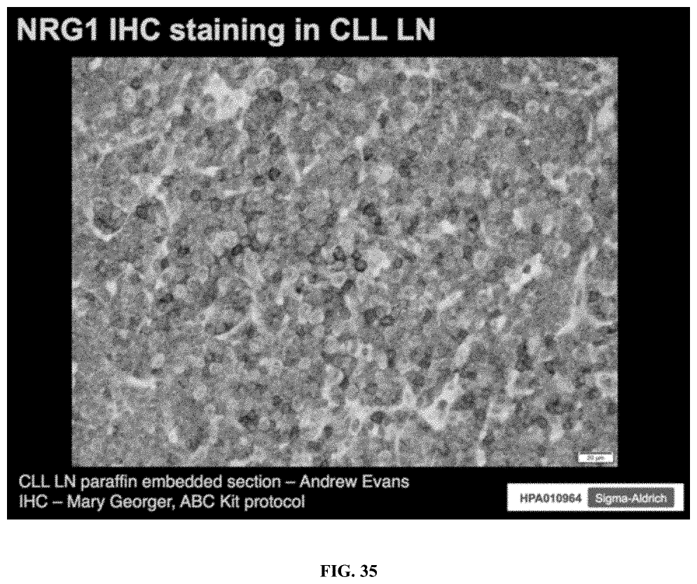

These data support that, among the EGFR family members, HER3 and HER4 are responsible for maintaining the viability of hematological malignant cells. Without wishing to be bound, a plausible explanation to the protein data is that HER3/HER4 dimers are responding to a ligand, resulting in HER4 cleavage producing the HER4 ICD fragment. The only reported ligands common for HER3 and HER4 are NRG1 and NRG2. Interestingly, in the Human Protein Atlas, NRG1 protein expression is detected by IHC primarily in lymphomas and no other cancer types (FIG. 34).

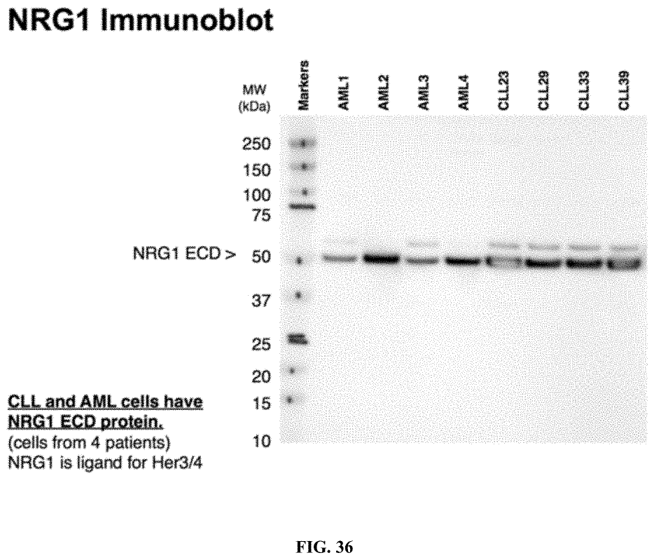

Presented herein is data from the Human Protein Atlas showing the EGFR family ligands are detectable in biopsies from NHL patients, including NRG1 (FIG. 23). Additional data is presented from experiments with CLL lymph node biopsies, showing moderate to strong NRG1 staining by IHC (FIG. 35). Moreover, NRG1 protein was readily detectable by immunoblot in protein extracts from primary AML and CLL cells (FIG. 36). Interestingly, all cells have the same the major detectable NRG1 protein products, which is a truncated fragment consistent with the NRG1 extracellular domain.

As demonstrated herein, inhibition of the EGFR family, in particular HER3 and HER4 members, can block the viability and are toxic to chronic lymphocytic leukemia cells (FIGS. 1-4), acute myeloid leukemia cells (FIGS. 6-7), and p53 mutated non-Hodgkin lymphoma cell lines [FIGS. 9-10]. Specifically, inhibition of EGFR by targeting the tyrosine kinase domain can exert lethal effects on these cells. Data presented herein show that HER3 and HER4 members of the EGFR family interact with their common ligands, such as NRG1, which is important for maintaining viability of hematological malignant cells, such as AML, CLL, and NHL.

The present invention thus enables a novel targeted therapy for hematological malignancies with the potential to provide major clinical benefits. Studies have demonstrated that EGFR inhibitors are largely safe and well tolerated. They may make hematologic malignancies more responsive to available therapies and may also be used to replace currently applied conventional chemotherapy regimens that are much more toxic and with uncertain efficacy.

For hematological malignancies, such as CLL, that are treated with other tyrosine kinase inhibitors such as BTK inhibitors, EGFR inhibitors may prove more effective. Additionally, targeting EGFR signaling may lead to therapies that result in long-term remissions and potentially cure these diseases as it targets a vital survival signal in the microenvironment of the cancer “stem” cell. Significantly, the present invention allows EGFR inhibitors to treat patients who have developed resistance to current standard therapies and target the most drug-resistant cancer “stem” cells that have been shown to be difficult to treat with currently available therapies.

Another unique feature of the invention is the repurposing of clinically available drugs (e.g., small molecule inhibitors of or monoclonal antibodies against the EGFR family, in particular HER3 and HER4 members) that inhibit EGFR family signaling, allowing such clinically available drugs to be quickly available to treat hematological malignancies.

Thus, in one aspect, the invention generally relates to a method for treating a hematologic malignancy, or a related disease or condition thereof. The method comprises administering to a subject in need thereof a pharmaceutical composition comprising an inhibitor of a member of the EGFR family, or a pharmaceutically acceptable salt, ester or pro-drug thereof.

In another aspect, the invention generally relates to a method for inhibiting or slowing tumor growth of a hematologic malignancy. The method includes administering to a subject in need thereof a pharmaceutical composition comprising an inhibitor of a member of epidermal growth factor receptor (EGFR) family, in particular HER3 and HER4 members, or a pharmaceutically acceptable salt, ester or pro-drug thereof.

As used herein, the term “epidermal growth factor receptor (EGFR)” or “ErbB1” or “HER1” refers to a transmembrane glycoprotein of 170 kDa that is encoded by the c-erbB1 proto-oncogene located in the 7q22 chromosome. EGFR is a member of the human epidermal growth factor receptor (EGFR) family of receptor tyrosine kinases (RTK) that includes HER2 (ErbB2), HER3 (ErbB3) and HER4 (ErbB4).

In certain embodiments, the inhibitor of a member of the EGFR family is an inhibitor of HER3. In certain embodiments, the inhibitor is an inhibitor of HER4.

In certain embodiments, the inhibitor of a member of the EGFR family is a small molecule compound. In certain embodiments, the small molecule inhibitor of a member of the EGFR family is selected from: afatinib (Gilotrif), lapatinib (Tykerb), neratinib (Nerlynx), gefitinib (Iressa), erlotinib (Tarceva), vandetanib (Caprelsa), osimertinib (Tagrisso), dacomitinib (Vizimpro). In certain embodiments, the small molecule compound is afatinib. Non-limiting examples of inhibitors of a member of the EGFR family includes those listed in Table 1.

```
TABLE 1
Examples of Inhibitors of EGFR Family Receptors
  Afatinib (Gilotrif)
  Lapatinib (Tykerb)
  Neratinib (Nerlynx)
  Gefitinib (Iressa)
  Erlotinib (Tarceva)
  Vandetanib (Caprelsa)
  Osimertinib (Tagrisso)
```

In certain embodiments, the inhibitor is an irreversible inhibitor that covalently binds to a member of the EGFR family, in particular HER3 or HER4.

In certain embodiments, the inhibitor is a reversible inhibitor that does not covalently bind to a member of the EGFR family, in particular HER3 or HER4.

In certain embodiments, the inhibitor a member of the EGFR family is a peptide. In certain embodiments, the inhibitor is an antibody. In certain preferred embodiments, the inhibitor is a monoclonal antibody (mAb). Non-limiting examples of mAbs include Trastuzumab (Herceptin), cetuximab (Erbitux), panitumumab (Vectibix), necitumumab (Portrazza), pertuzumab (Omnitarg).

In certain embodiments, administration to the subject is via oral, intravenous, intramuscular, or subcutaneous administration.

In certain preferred embodiments when the inhibitor is a small molecule, administration to the subject is via oral administration. In certain preferred embodiments when the inhibitor is a peptide (e.g., antibody), administration to the subject is via intravenous, intramuscular, or subcutaneous administration.

Any suitable hematologic malignancies, or related diseases and conditions, may be treated by the method disclosed herein, for example, leukemia, non-Hodgkin lymphoma, Hodgkin lymphoma, multiple myeloma, and myelodysplastic syndromes.

In certain embodiments, the hematologic malignancy is leukemia. In certain embodiments, the hematologic malignancy is AML. In certain embodiments, the hematologic malignancy is CIVIL. In certain embodiments, the hematologic malignancy is ALL. In certain embodiments, the hematologic malignancy is CLL.

In certain embodiments, the method further includes administering to the subject one or more other anti-cancer agents.

In certain embodiments, the one or more other anti-cancer agents include a chemotherapeutic agent. Any suitable chemotherapeutic agent may be employed, for example, one or more selected from cyclophosphamide, doxorubicin, bendamustine, cytarabine, mitoxantrone, adriamycin, etoposide, prednisone, vincristine, methotrexate and fludarabine.

In certain embodiments, the one or more other anti-cancer agents include mAbs, for example, anti-CD20 mAbs or anti-CD38 mAbs (e.g., Daratumumab). Any suitable anti-CD20 mAbs may be employed, for examples, rituximab (Rituxan), obinutuzumab (Gazyva), ofatumumab (Arzerra). Drugs linked to mAbs, such as gemtuzumab ozogamicin (Mylotarg), may also be used in combination.

In certain embodiments, the method further includes administering to the subject include a chemotherapeutic agent and a therapeutic mAb (e.g., an anti-CD20 or anti-CD38 mAbs).

As first disclosed herein, the method of the invention may be employed to treat a subject that has had prior treatment with another anti-cancer agent which is not an inhibitor of a member of the EGFR family, in particular HER3 or HER4.

In certain embodiments, the subject has developed resistance to the other anti-cancer agent.

In certain embodiments, the other anti-cancer agent is a Bruton's tyrosine kinase (BTK) inhibitor, for example ibrutinib or acalabrutinb.

In yet another aspect, the invention generally relates to a pharmaceutical composition comprising an inhibitor of a member of the EGFR family, in particular HER3 or HER4, or a pharmaceutically acceptable salt, ester or pro-drug thereof, in an amount effective in the treatment of a hematologic malignancy, or a related disease or condition thereof, in a mammal, including a human, and a pharmaceutically acceptable excipient, carrier, or diluent.

In yet another aspect, the invention generally relates to a pharmaceutical composition comprising an inhibitor of a member of the EGFR family, in particular HER3 or HER4, or a pharmaceutically acceptable salt, ester or pro-drug thereof, in an amount effective in inhibiting or slowing tumor growth of a hematologic malignancy, or a related disease or condition thereof, in a mammal, including a human, and a pharmaceutically acceptable excipient, carrier, or diluent.

In certain embodiments of the pharmaceutical composition, the inhibitor of EGFR is a small molecule compound. Non-limiting examples of small molecule inhibitors of a member of the EGFR family include: afatinib (Gilotrif), lapatinib (Tykerb), neratinib (Nerlynx), gefitinib (Iressa), erlotinib (Tarceva), vandetanib (Caprelsa), osimertinib (Tagrisso), dacomitinib (Vizimpro).

In certain preferred embodiments, the small molecule compound is afatinib.

In certain embodiments, the inhibitor is a peptide. In certain embodiments, the inhibitor is an antibody. Non-limiting examples of mAbs include Trastuzumab (Herceptin), cetuximab (Erbitux), panitumumab (Vectibix), necitumumab (Portrazza), pertuzumab (Omnitarg).

In certain embodiments, the pharmaceutical composition is suitable for oral administration. In certain embodiments, the pharmaceutical composition is suitable for one or more of intravenous, intramuscular, and subcutaneous administration.

In certain embodiments, the pharmaceutical composition is effective to treat one or more hematologic malignancies selected from leukemia, non-Hodgkin lymphoma, Hodgkin lymphoma, multiple myeloma, and myelodysplastic syndromes.

In certain embodiments, the pharmaceutical composition is effective to treat leukemia. In certain embodiments, the pharmaceutical composition is effective to treat AML. In certain embodiments, the pharmaceutical composition is effective to treat CIVIL. In certain embodiments, the pharmaceutical composition is effective to treat ALL. In certain embodiments, the pharmaceutical composition is effective to treat CLL.

In yet another aspect, the invention generally relates to a unit dosage form that comprises a compound or a pharmaceutical composition disclosed herein.

The unit dosage form may be in any suitable form. In certain embodiments, the unit dosage form is in the form of a tablet or capsule suitable for oral administration.

In certain embodiments, the unit dosage form is in the form of a liquid solution or suspension suitable for intravenous, intramuscular or subcutaneous administration.

In yet another aspect, the invention generally relates to a method for affecting a microenvironmental factor of a hematological cancer cell. The method includes administering to a subject suffering from a hematologic malignancy, or a related disease or condition thereof, an inhibitor of a member of the EGFR family, in particular HER3 or HER4, or a pharmaceutically acceptable salt, ester or pro-drug thereof.

In certain embodiments of the method, the hematologic malignancy is selected from leukemia, non-Hodgkin lymphoma, Hodgkin lymphoma, multiple myeloma, myelodysplastic syndromes, myeloproliferative disorders such as essential thrombocythemia, polycythemia vera, and primary myelofibrosis.

Isotopically-labeled compounds are also within the scope of the present disclosure. As used herein, an “isotopically-labeled compound” refers to a presently disclosed compound including pharmaceutical salts and prodrugs thereof, each as described herein, in which one or more atoms are replaced by an atom having an atomic mass or mass number different from the atomic mass or mass number usually found in nature. Examples of isotopes that can be incorporated into compounds presently disclosed include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

By isotopically-labeling the presently disclosed compounds, the compounds may be useful in drug and/or substrate tissue distribution assays. Tritiated (³H) and carbon-14 (¹⁴C) labeled compounds are particularly preferred for their ease of preparation and detectability. Further, substitution with heavier isotopes such as deuterium (²H) can afford certain therapeutic advantages resulting from greater metabolic stability, for example increased in vivo half-life or reduced dosage requirements and, hence, may be preferred in some circumstances. Isotopically labeled compounds presently disclosed, including pharmaceutical salts, esters, and prodrugs thereof, can be prepared by any means known in the art.

Further, substitution of normally abundant hydrogen (¹H) with heavier isotopes such as deuterium can afford certain therapeutic advantages, e.g., resulting from improved absorption, distribution, metabolism and/or excretion (ADME) properties, creating drugs with improved efficacy, safety, and/or tolerability. Benefits may also be obtained from replacement of normally abundant ¹²C with ¹³C. (See, WO 2007/005643, WO 2007/005644, WO 2007/016361, and WO 2007/016431.)

Stereoisomers (e.g., cis and trans isomers) and all optical isomers of a presently disclosed compound (e.g., R and S enantiomers), as well as racemic, diastereomeric and other mixtures of such isomers are within the scope of the present disclosure.

Compounds of the present invention are, subsequent to their preparation, preferably isolated and purified to obtain a composition containing an amount by weight equal to or greater than 95% (“substantially pure”), which is then used or formulated as described herein. In certain embodiments, the compounds of the present invention are more than 99% pure.

Solvates and polymorphs of the compounds of the invention are also contemplated herein. Solvates of the compounds of the present invention include, for example, hydrates.

Any appropriate route of administration can be employed, for example, parenteral, intravenous, subcutaneous, intramuscular, intraventricular, intracorporeal, intraperitoneal, rectal, or oral administration. Most suitable means of administration for a particular patient will depend on the nature and severity of the disease or condition being treated or the nature of the therapy being used and on the nature of the active compound.

Solid dosage forms for oral administration include capsules, tablets, pills, powders, and granules. In such solid dosage forms, the compounds described herein or derivatives thereof are admixed with at least one inert customary excipient (or carrier) such as sodium citrate or dicalcium phosphate or (i) fillers or extenders, as for example, starches, lactose, sucrose, glucose, mannitol, and silicic acid, (ii) binders, as for example, carboxymethylcellulose, alignates, gelatin, polyvinylpyrrolidone, sucrose, and acacia, (iii) humectants, as for example, glycerol, (iv) disintegrating agents, as for example, agar-agar, calcium carbonate, potato or tapioca starch, alginic acid, certain complex silicates, and sodium carbonate, (v) solution retarders, as for example, paraffin, (vi) absorption accelerators, as for example, quaternary ammonium compounds, (vii) wetting agents, as for example, cetyl alcohol, and glycerol monostearate, (viii) adsorbents, as for example, kaolin and bentonite, and (ix) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents. Solid compositions of a similar type may also be employed as fillers in soft and hard-filled gelatin capsules using such excipients as lactose or milk sugar as well as high molecular weight polyethyleneglycols, and the like. Solid dosage forms such as tablets, dragees, capsules, pills, and granules can be prepared with coatings and shells, such as enteric coatings and others known in the art.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, such as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like. Besides such inert diluents, the composition can also include additional agents, such as wetting, emulsifying, suspending, sweetening, flavoring, or perfuming agents.

Materials, compositions, and components disclosed herein can be used for, can be used in conjunction with, can be used in preparation for, or are products of the disclosed methods and compositions. It is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

The following examples are meant to be illustrative of the practice of the invention, and not limiting in any way.

### EXAMPLES

### CLL Cells Lose Viability and are Killed by an EGFR Family Inhibitor In Vitro

Tests of CLL cell viability after BTK inhibitor treatment showed that relatively high doses of BTK inhibitors are needed to decrease CLL cell viability in vitro, indicating that a common off-target effect of these inhibitors may be responsible. The common off-target with the best binding was ERRB2 in the EGFR family of receptors. Afatinib (an irreversible inhibitor that covalently binds to the EGFR family of receptors (including ERBB2) active tyrosine kinase domain ATP-binding site, a similar mechanism to that of the BTK inhibitors) was chosen as an example EGFR family inhibitor to test this hypothesis. CLL patient cells (1×10⁵ cells/100 μL media) were cultured for 48 hours in clear 96-well flat bottom tissue culture plates (Costar, Corning, Corning, N.Y.) with serial drug dilutions (100 μM afatinib (LC Laboratories, Woburn, Mass.) serially diluted 3-fold ending at ˜0.01 μM). Media is RPMI1640 (Cellgro, Corning) supplemented with penicillin, streptomycin, L-glutamine (Gibco, ThermoFisher Scientific, Waltham, Mass.), and 10% heat-inactivated fetal bovine serum (Sigma-Aldrich, St. Louis, Mo.). After cell culture, viability was measured by Cell Titer-Blue Cell Viability Assay (Promega, Madison, Wis.) according to manufacturer's directions. Non-viable cells produce less fluorescence. Measurements were done in triplicate for each concentration. Fluorescence was measured on a BioTek Synergy2 96-well plate reader (BioTek Instruments, Winooski, Vt.). Data was analysed using Prism6 software (GraphPad Software, La Jolla, Calif.) and dose curve was generated using 4PL fit. Afatinib reduces CLL cell viability in a dose dependent manner (IC50 0.4 μM) as shown in FIG. 1 (all data points shown).

To confirm that an EGFR family inhibitor reduces CLL cell viability, another viability assay was used to measure the effect of afatinib on CLL cells in vitro. The previous experiment was repeated and cell viability was measured by the Cell Titer-Glo 2.0 Assay (Promega). Non-viable cells produce less luminescence. After cell culture, viability was assayed according to manufacturer's directions. Luminescence was measured on a BioTek Synergy2 96-well plate reader (BioTek Instruments). Data was analysed using Prism6 software (GraphPad Software) and dose curve was generated using 4PL fit. The data confirmed that afatinib reduces CLL cell viability in a dose dependent manner (IC50 0.3 μM) as shown in FIG. 2 (all data points shown).

To test that an EGFR family inhibitor not only reduces CLL cell viability, but also induces cell death in vitro, cytotoxicity was measured by CellTox Green Cytotoxicity Assay (Promega). Dead cells produce more fluorescence in this assay. CLL patient cells (1×10⁵ cells/100 μL media) were cultured as in the previous experiment. After cell culture, cytotoxicity was assayed according to manufacturer's directions. Fluorescence was measured on a BioTek Synergy2 96-well plate reader (BioTek Instruments). Data was analysed using Prism6 software (GraphPad Software) and dose curve was generated using 4PL fit. Based on this data, afatinib induces CLL cell death in a dose dependent manner (IC50 12 μM) as shown in FIG. 3 (all data points shown).

To compare an EGFR family inhibitor to a BTK inhibitor in their ability to reduce CLL cell viability, the previous experiment was repeated with both inhibitors. Cell viability was measured by Cell Titer-Glo 2.0 Assay (Promega) in triplicate for each concentration. Afatinib and ibrutinib were chosen as the EGFR family inhibitor and BTK inhibitor, respectively. CLL patient cells (1×10⁵ cells/100 μL media) were cultured for 48 hours in clear 96-well flat bottom tissue culture plates (Costar, Corning) with serial drug dilutions (100 μM AFA (LC Laboratories) or IBR (Selleckchem, Houston, Tex.) serially diluted 3-fold ending at ˜0.01 μM). Media was RPMI1640 (Cellgro, Corning) supplemented with penicillin, streptomycin, L-glutamine (Gibco, ThermoFisher Scientific), and 10% heat-inactivated fetal bovine serum (Sigma-Aldrich). After cell culture, viability was assayed according to manufacturer's directions. Luminescence was measured on a BioTek Synergy2 96-well plate reader (BioTek Instruments). Data was analysed using Prism6 software (GraphPad Software) and dose curve was generated using 4PL fit. These data demonstrate that afatinib reduces CLL cell viability (IC50 0.5 μM) at a 20-fold lower concentration than ibrutinib (IC50 10 μM) as shown in FIG. 4 (mean with standard deviation error bars shown).

### RNA Expression in AML Cells Suggests an Important Role for EGFR Family Member Signaling in Survival of AML “Stem” Cells

Myeloid malignancies, such as AML are difficult to treat because the AML cancer “stem” cells are particularly insensitive to current therapies. The present studies with an AML tumor microenvironment model indicated that EGFR signaling was vital to the survival of AML cancer stem cells. Primary human AML cells were seeded in a matrix that promotes some AML cell to become adherent with increased survival and chemotherapy resistance, resembling cancer “stem” cells. Cancer “stem” cells can self-renew and give rise to all the tumor cell types. Cancer “stem” cells are generally resistant to current therapy and therapeutic interventions that target these cells are needed in order to realize possible cures. The present studies found that these AML adherent cells significantly upregulate FAM83A and MIR34A gene expression compared to suspension AML cells by RNA-Seq analysis. FAM83A promotes EGFR family signaling, leading to PI3K-AKT-mTOR and MAPK signaling cascades. This signaling leads to a switch from oxidative phosphorylation to glycolysis (possibly through HIF-1alpha). The change in metabolism to glycolysis is a key feature of cancer cells (Warburg effect). These data have led to the present AML model (FIG. 5), indicating an important role for the EGFR family in the survival of AML “stem” cells.

### AML Cells Lose Viability and are Killed by an EGFR Family Inhibitor In Vitro

Based on the present AML model (FIG. 5), adherent AML cells are resistant to current therapy due to EGFR family signaling. Furthermore, non-adherent suspension AML cells may be especially vulnerable to EGFR family signal inhibition, because these cells have left the tumor microenvironment and are no longer receiving maximal EGFR family signals. To test this, primary AML suspension cells were treated in vitro with afatinib. Cell viability was measured by Cell Titer-Glo 2.0 Assay (Promega) in triplicate for each concentration. Two different AML patient cells (1×10⁵ cells/100 μL media) were cultured for 30 hours in clear 96-well flat bottom tissue culture plates (Costar, Corning) with serial drug dilutions (100 μM afatinib (LC Laboratories) serially diluted 3-fold ending at ˜0.01 μM). Media is RPMI1640 (Cellgro, Corning) supplemented with penicillin, streptomycin, L-glutamine (Gibco, ThermoFisher Scientific), and 10% heat-inactivated fetal bovine serum (Sigma-Aldrich). After cell culture, viability was assayed according to manufacturer's directions. Luminescence was measured on a BioTek Synergy2 96-well plate reader (BioTek Instruments). Data was analysed using Prism6 software (GraphPad Software) and dose curve was generated using 4PL fit. Afatinib reduces cell viability in two different AML patient samples in a dose dependent manner (IC50 2.1 and 2.6 μM) as shown in FIG. 6 (mean with standard deviation error bars shown).

To test that an EGFR family inhibitor not only reduces AML cell viability, but also induces cell death in vitro, cytotoxicity was measured by CellTox Green Cytotoxicity Assay (Promega). One AML patient cells (1×10⁵ cells/100 μl media) were cultured as in the previous experiment. After cell culture, cytotoxicity was assayed according to manufacturer's directions. Fluorescence was measured on a BioTek Synergy2 96-well plate reader (BioTek Instruments). Data was analysed using Prism6 software (GraphPad Software) and dose curve was generated using 4PL fit. Afatinib induces AML cell death in a dose dependent manner (IC50 19 μM) as shown in FIG. 7 (all data points shown).

### General Model of Cancer Cell and EGFR Family Interactions in the Tumor Microenvironment of Hematological Malignancies.

Based on these results from CLL and AML cells, the present inventors developed a general model of cancer cell and EGFR family interactions in the tumor microenvironment of hematological malignancies (FIG. 8). The tumor microenvironment can be from a lymphoid or myeloid malignancy. The cancer cell can have properties of cancer “stem” cells, such as chemoresistance, which may be due in part to EGFR family of receptor signals in the tumor microenvironment. EGFR family of receptors are illustrated as EGFR dimer, which may be EGFR, ERRB2, ERRB3, ERBB4 homodimers or heterodimers. EGFR family of receptor ligands are illustrated as membrane bound or soluble EGFR ligand, which may be EGF, AREG, BTC, EPGN, EREG, HBEGF, TGFA, NRG1, NRG2, NRG3, NRG4, or as yet unreported EGFR family of receptor ligands. These ligands are available in the microenvironment as soluble factors, extracellular matrix-bound factors, or membrane-bound factors expressed on the surface of stromal cells. The resulting EGFR family of receptors signaling provides survival advantages for the cancer cell in this environment. This model makes several predictions: 1) Blocking EGFR family signaling would result in loss of viability and cell death of the cancer cell. Data disclosed herein have shown this for CLL, AML and NHL cells or cell lines. 2) Blocking the EGFR family signaling is not dependent on the p53 pathway. Therefore, especially aggressive p53 mutant cancers may still be susceptible to therapies that block EGFR family signaling. 3) Hematological malignant cells will express EGFR family members. 4) Cells in the tumor microenvironment of hematological malignancies will express EGFR family ligands.

### NHL p53 Mutated Cell Line Loses Viability and is Killed by an EGFR Family Inhibitor In Vitro

One prediction of the general model disclosed herein (FIG. 8) is that inhibition of EGFR family signaling will generally decrease viability of many types of hematological malignancies including those that are p53 mutated. NHL is the predominant class of lymphoma worldwide. There are many types of NHL. BL is one particularly aggressive type of NHL. This type of NHL-derived cell line was chosen to test with EGFR family signaling inhibition. The BL-derived CA46 cell line also contains a homozygous p53 mutation, which will also enable us to test if EGFR inhibition requires an intact p53 pathway. The CA46 cell line (ATCC CRL-1648, p.R248Q homozygous p53 mutation, BL-derived) (1×10⁴ cells/100 μL media) was cultured for 54 hours in clear 96-well flat bottom tissue culture plates (Costar, Corning) with serial drug dilutions (100 μM afatinib (LC Laboratories) or dexamethasone (ThermoFisher Scientific) serially diluted 3-fold ending at ˜0.01 μM) in triplicate for each concentration. Dexamethasone (DEX) was chosen as a drug comparison because DEX is a cytotoxic drug for lymphoid malignancies and is part of the current chemotherapy regimen for BL. Media was RPMI1640 (Cellgro, Corning) supplemented with penicillin, streptomycin, L-glutamine (Gibco, ThermoFisher Scientific), and 10% heat-inactivated fetal bovine serum (Sigma-Aldrich). After cell culture, viability was measured by Cell Titer-Glo 2.0 Assay (Promega) according to manufacturer's directions. Luminescence was measured on a BioTek Synergy2 96-well plate reader (BioTek Instruments). Data was analysed using Prism6 software (GraphPad Software) and dose curve was generated using 4PL fit. Afatinib reduces cell viability of this p53 mutated Non-Hodgkin Lymphoma (NHL) cell line in a dose dependent manner (3 μM), whereas dexamethasone (DEX) does not as shown in FIG. 9 (mean with standard deviation error bars shown).

To test that an EGFR family inhibitor not only reduces cell viability in an NHL p53 mutated cell line, but also induces cell death in vitro, cytotoxicity was measured by CellTox Green Cytotoxicity Assay (Promega). The CA46 cell line (ATCC) (1×10⁴ cells/100 μL media) was cultured as in the previous experiment. After cell culture, cytotoxicity was assayed according to manufacturer's directions. Fluorescence was measured on a BioTek Synergy2 96-well plate reader (BioTek Instruments). Data was analysed using Prism6 software (GraphPad Software) and dose curve was generated using 4PL fit. Afatinib induces cell death of this p53 mutated NHL cell line in a dose dependent manner (22 μM), whereas dexamethasone (DEX) does not as shown in FIG. 10 (mean with standard deviation error bars shown).

### EGFR Family Gene Expression in Hematological Malignancies

A second prediction of the general model (FIG. 8) is that EGFR family gene expression will be found in hematological malignancies. To test this hypothesis, the present inventors mined publicly available microarray data for RNA expression of the EGFR family (EGFR, ERBB2, ERBB3, ERBB4). These data were analyzed and visualized using the R2: Genomics Analysis and Visualization Platform (http://r2.amc.nl). Available hematological malignancy microarray data include CLL, AML, BL, FL, DLBCL, MCL, myeloma, and ALL. The EGFR family is expressed in CLL cells (FIG. 11). Two representative CLL microarray datasets are shown. Expression of all EGFR family members is present in CLL, with high expression of ERBB2 consistent between the two datasets.

To test RNA expression of the EGFR family in AML, the present inventors mined publicly available microarray data as above. The EGFR family is highly expressed in AML cells (FIG. 12). One representative AML dataset is shown.

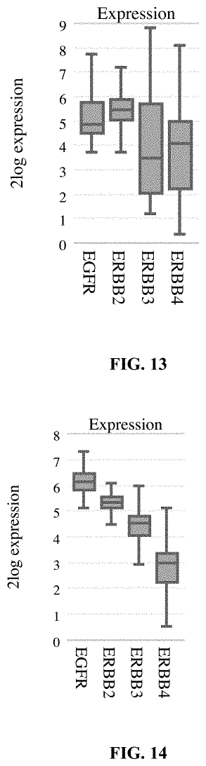

To test RNA expression of the EGFR family in BL, the present inventors mined publicly available microarray data as above. The EGFR family is highly expressed in BL cells (FIG. 13). One representative BL dataset is shown.

To test RNA expression of the EGFR family in FL, the present inventors mined publicly available microarray data as above. The EGFR family is variably expressed in FL cells (FIG. 14). All EGFR family members are expressed in FL. One representative FL dataset is shown.

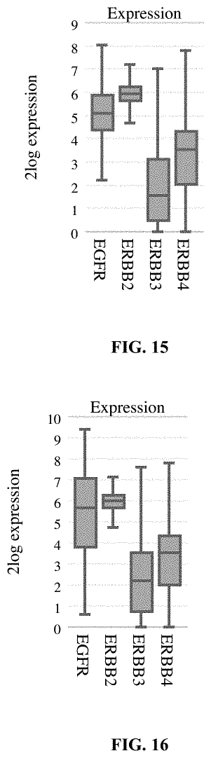

To test RNA expression of the EGFR family in DLBCL, the present inventors mined publicly available microarray data as above. The EGFR family is variably expressed in DLBCL cells (FIG. 15). All EGFR family members are typically expressed in DLBCL. ERBB2 is consistently high among DLBCL samples. One representative DLBCL dataset is shown.

To test RNA expression of the EGFR family in MCL, the present inventors mined publicly available microarray data as above. The EGFR family is variably expressed in MCL cells (FIG. 16). All EGFR family members are typically expressed in MCL. ERBB2 is consistently high among MCL samples. One representative MCL dataset is shown.

To test RNA expression of the EGFR family in myeloma, the present inventors mined publicly available microarray data as above. The EGFR family is highly expressed in myeloma cells (FIG. 17). One representative myeloma dataset is shown.

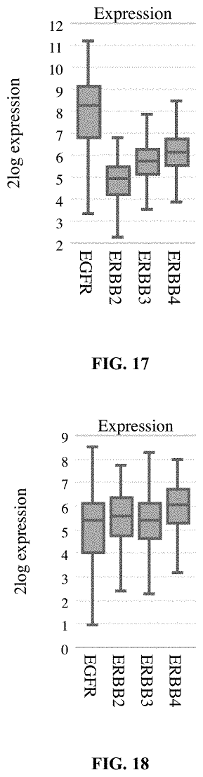

To test RNA expression of the EGFR family in ALL, the present inventors mined publicly available microarray data as above. The EGFR family is highly expressed in ALL cells (FIG. 18). One representative ALL dataset is shown.

### EGFR Family Ligands RNA Expression in Hematological Malignancies

A third prediction of the general model (FIG. 8) is that EGFR family ligands gene expression will be found in the microenvironment of hematological malignancies. To test this hypothesis, the present inventors mined publicly available microarray data for RNA expression of the EGFR family ligands (EGF, AREG, BTC, EPGN, EREG, HBEGF, TGFA, NRG1, NRG2, NRG3, NRG4). These data were analyzed and visualized using the R2: Genomics Analysis and Visualization Platform (http://r2.ame.nl). Much of the publicly available microarray data utilizes purified cells to provide clearer results about specific cell populations. However, for many lymphomas, purified cells are difficult to obtain, and often tissue biopsies with a majority of tumor cells are assessed. These tissue biopsy results provide gene expression data of the tumor and surrounding microenvironment and were mined for EGFR family ligands gene expression to provide evidence of possible microenvironment production. Available tumor microarray data from hematological malignancy biopsies include BL, FL, DLBCL, and MCL sample. BL microarray data obtained from lymph node biopsies demonstrated expression of EGFR family ligands (except EPGN was not available for testing in these microarrays) as shown in FIG. 19.

Microarray data from FL biopsies demonstrated RNA expression of EGFR family ligands (except EPGN was not available for testing in these microarrays) as shown in FIG. 20.

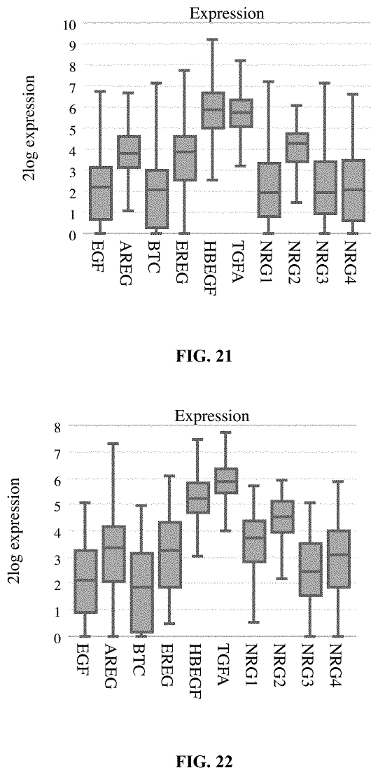

Microarray data from DLBCL biopsies demonstrated RNA expression of EGFR family ligands (except EPGN was not available for testing in these microarrays) as shown in FIG. 21.

Microarray data from MCL biopsies demonstrated RNA expression of EGFR family ligands (except EPGN was not available for testing in these microarrays) as shown in FIG. 22. EGFR family ligands protein expression in hematological malignancies

To further support the prediction that EGFR family ligands gene expression will be found in the microenvironment of hematological malignancies, the present inventors mined publicly available immunohistochemically stained lymphoma tissue samples for protein expression of the EGFR family ligands (EGF, AREG, BTC, EPGN, EREG, HBEGF, TGFA, NRG1, NRG2, NRG3, NRG4). These data were made available and visualized using the Human Protein Atlas (http://www.proteinatlas.org) (M. Uhlen et al., Science 357, eaan2507 (2017). DOI: 10.1126/science.aan2507). The degree of brown staining indicates the presence of an antibody specifically recognized protein. Antibody stainings of AREG, HBEGF, TGFA, NRG1, NRG2, NRG4 in lymphoma pathology specimens were available. NRG1 staining of NHL tissue specimens was especially high and strong intensity. A representative staining is shown in FIG. 23. Membranous and diffuse non-tumor cell staining could be associated with NRG1 protein on surrounding extracellular matrix or stromal cells.

Immunohistochemistry staining with NRG2 antibody of NHL tissue specimens exhibited moderate low intensity staining as shown by a representative sample in FIG. 24. Diffuse non-tumor cell staining could be associated with NRG2 protein on surrounding extracellular matrix or stromal cells.

Immunohistochemistry staining with NRG4 antibody of NHL tissue specimens exhibited weak cytoplasmic and membranous staining as shown by a representative sample in FIG. 25. This staining appears to be mostly non-tumor cell, suggesting that NRG4 protein is mostly on surrounding extracellular matrix or stromal cells.

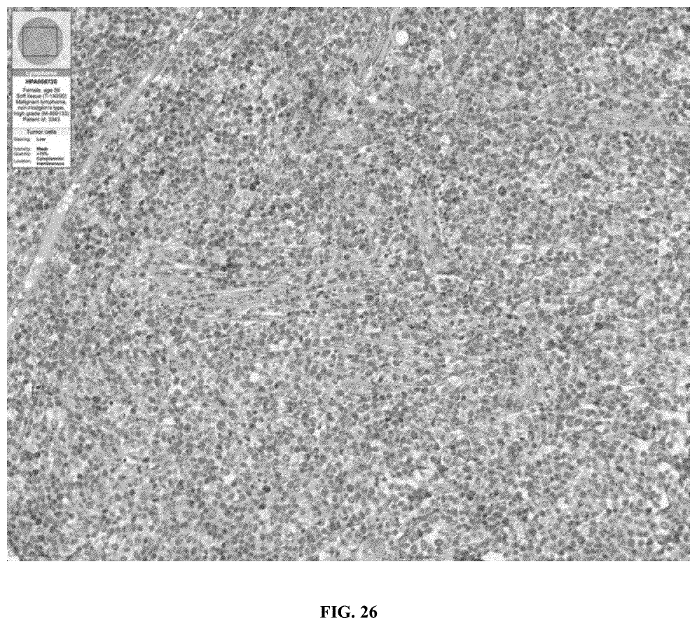

Immunohistochemistry staining with AREG antibody of NHL tissue specimens exhibited weak cytoplasmic and membranous staining as shown by a representative sample in FIG. 26. This staining appears to be on tumor and non-tumor material and cells, suggesting that AREG protein can be found on surrounding extracellular matrix or stromal cells.

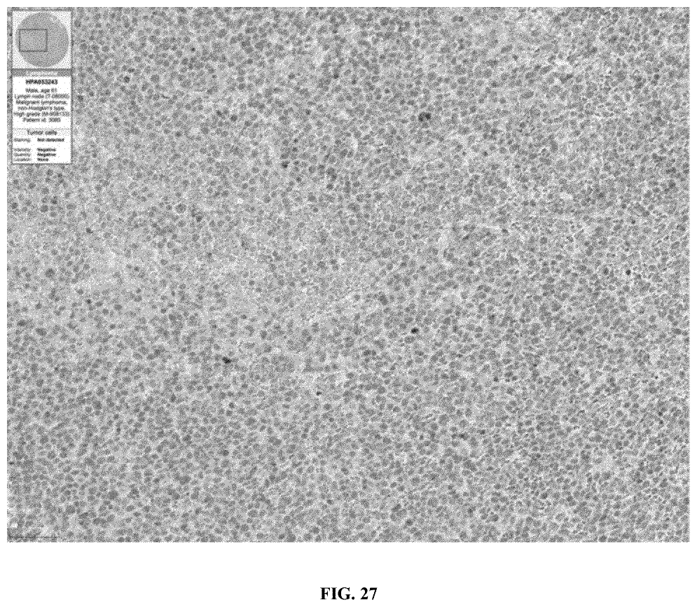

Immunohistochemistry staining with HBEGF antibody of NHL tissue specimens was not detected on tumor cells, but weak cytoplasmic and membranous staining was detectable as shown by a representative sample in FIG. 27. This staining appears to be non-tumor material and cells, suggesting that HBEGF protein can be found on surrounding extracellular matrix or stromal cells.

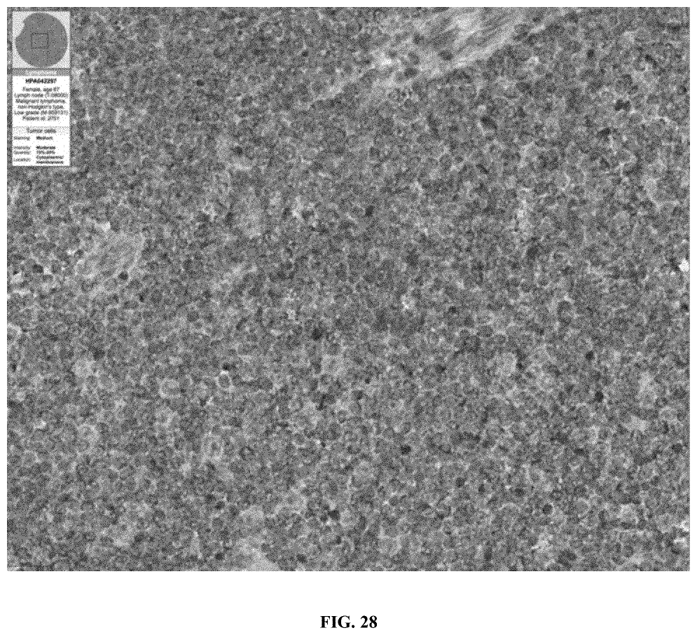

Immunohistochemistry staining with TGFA antibody of NHL tissue specimens exhibited moderate cytoplasmic and membranous staining as shown by a representative sample in FIG. 28. Membranous and diffuse non-tumor cell staining could be associated with TGFA protein on surrounding extracellular matrix or stromal cells.

### CLL Animal Model Testing of EGFR Inhibitor Efficacy

EGFR inhibition of CLL is tested in a patient-derived xenograft (PDX) model. (Herman et al. 2017 Clin. Cancer Res. 23(11):2831-41.) In this model, CLL patient peripheral blood mononuclear cells from an established CLL tissue bank is injected intravenously into NSG (NOD-scid IL2Rγ^{null}) mice resulting in CLL cell engraftment and proliferation after about 3-4 weeks. Six different CLL patient samples are tested to account for patient variability. Each sample is injected into 10 mice to ensure CLL engraftment is obtained for each condition (5 mice with drug, 5 mice with vehicle). For the EGFR inhibitor drug, afatinib is used. Blood samples are acquired every other week to monitor the leukemia. After 3-4 weeks, mice are sacrificed and measures of CLL engraftment and proliferation are performed.

### AML Animal Model Testing of EGFR Inhibitor Efficacy

Afatinib inhibition of AML is tested in two animal models, PDX AML mouse model and the MLL-AF9 mouse model.

The PDX AML mouse model is the current principal AML mouse model for drug testing. (Townsend et al. 2016 Cancer Cell. 29(4):574-86.) This model involves injection of patient mononuclear cells either from blood or bone marrow in NSG mice resulting in AML after 4-8 weeks that has incomplete penetrance. These injections may be performed onsite, or alternatively, AML xenograft mouse models are acquired that have well-characterized AML cells regarding their genetics, as well as their percentage penetrance in NSG mouse strains.

The PDX mouse models are very useful because drugs can be tested against actual human cancer cells in vivo. However, PDX models lack a functioning adaptive immune system. In the MLL-AF9 mouse model, the MLL-AF9 oncogene has been introduced by homologous recombination and results in a disease distribution similar to adult MLL-AF9 leukemia, with the mice primarily succumbing to AML. This model is very useful because the penetrance of AML disease is very high and these animals can be drug-tested in a background of a normal immune system.

FIGS. 29 and 30 show HER3 protein expression in a representative NHL (Non-Hodgkin Lymphoma) tissue biopsy and a representative CLL (chronic lymphocytic leukemia) lymph node (LN) biopsy. Representative NHL tissue biopsy was stained with HPA045396, a rabbit polyclonal antibody that recognizes HER3. Type of NHL is indicated. Data is available at Human Protein Atlas website: http://www.proteinatlas.org. HER3 protein was expressed in NHL tissues. Representative CLL lymph node biopsy was stained with D22C5 (CellSignalingTechnologies #12708), a rabbit monoclonal antibody that recognizes HER3. CLL LN section obtained from URMC Pathology by Dr. Andrew Evans was stained per manufacturer directions by Mary Georger. HER3 protein was detectable in CLL cells residing in lymph node.

As demonstrated in FIG. 31, full length HER3 protein was detectable in AML (acute myeloid leukemia) primary cell extracts, either from suspension cells grown in tissue culture media or from suspension cells isolated from a tissue culture matrix or from adherent cells isolated from a tissue culture matrix.

Primary AML cells were cultured in tissue culture media with or without Decellularized Wharton's Jelly Matrix (DWJM) for 3 days and suspension cells were isolated and subsequently adherent cells were isolated from collagenase-treated DWJM. Protein was extracted from these cell preparations, denatured, size-separated by SDS-PAGE, and immunoblotted with D22C5 (CellSignalingTechnologies #12708), a rabbit monoclonal antibody that recognizes HER3.

FIG. 32 shows that full length HER3 protein was detectable in CLL (chronic lymphocytic leukemia) primary cells, as suspension cells grown in serum-free tissue culture media (AimV). Primary CLL cells were cultured overnight in serum-free tissue culture media (AimV). Protein was extracted from these cells, denatured, size-separated by SDS-PAGE, and immunoblotted with D22C5 (CellSignalingTechnologies #12708), a rabbit monoclonal antibody that recognizes HER3.

Shorter than full-length HER4 protein was detectable in AML (n=4) and CLL (n=4) primary cells, as suspension cells prepared from frozen aliquots, as shown in FIG. 33. The size is similar to that expected for just the HER4 intracellular domain (ICD) fragment.

Primary AML (n=4) cells and primary CLL (n=4) cells were prepared as suspension cells from frozen aliquots. Protein was extracted from these cells, denatured, size-separated by SDS-PAGE, and immunoblotted with 111B2 (Cell SignalingTechnologies #4795), a rabbit monoclonal antibody that recognizes HER4. The 111B2 mAb recognized a carboxy-terminal epitope of HER4, which is located in the ICD.

NRG1 protein expression is associated with lymphomas. As shown in FIG. 34, NRG1 protein staining was moderate to strong cytoplasmic staining in most lymphomas, but negative in remaining cancer cell types. Protein expression summary for cancer pathology specimens stained for NRG1. Only lymphoma specimens display NRG staining among all cancer subtypes. Data is available at Human Protein Atlas website: http://www.proteinatlas.org.

FIG. 35 shows NRG1 protein is detectable in CLL cells residing in lymph node. Representative CLL lymph node biopsy was stained with a rabbit polyclonal antibody that recognizes NRG1 (Sigma-Aldrich HPA010964). CLL LN section obtained from URMC Pathology by Dr. Andrew Evans was stained per manufacturer directions by Mary Georger.

FIG. 36 shows that shorter than full-length NRG1 protein was detectable in AML (n=4) and CLL (n=4) primary cells, as suspension cells prepared from frozen aliquots. The size is similar to that expected for the NRG1 extracellular domain (ECD) fragment. Primary AML (n=4) cells and primary CLL (n=4) cells were prepared as suspension cells from frozen aliquots. Protein was extracted from these cells, denatured, size-separated by SDS-PAGE, and immunoblotted with a rabbit polyclonal antibody (Sigma-Aldrich HPA010964) that recognizes the NRG1 extracellular domain (ECD). Shorter than full-length NRG1 protein is detectable in AML (n=4) and CLL (n=4) primary cells. The size is similar to that expected for a NRG1 extracellular domain (ECD) fragment. The polyclonal antibody recognizes amino-terminal sequence of NRG1, which is located in the ECD.

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples disclosed herein are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. The examples herein contain important additional information, exemplification and guidance that can be adapted to the practice of this invention in its various embodiments and equivalents thereof.

1. A method for treating a hematologic malignancy, or a related disease or condition thereof, comprising administering to a subject in need thereof a pharmaceutical composition comprising an inhibitor of a member of epidermal growth factor receptor (EGFR) family, or a pharmaceutically acceptable salt, ester or pro-drug thereof.

2. A method for inhibiting or slowing tumor growth of a hematologic malignancy, comprising administering to a subject in need thereof a pharmaceutical composition comprising an inhibitor of a member of epidermal growth factor receptor (EGFR) family, or a pharmaceutically acceptable salt, ester or pro-drug thereof.

3. The method of claim 1, wherein the inhibitor is an inhibitor of HER3 or HER4.

4. The method of claim 2, wherein the inhibitor is an inhibitor of HER3 or HER4.

5. The method of claim 1, wherein the inhibitor is a small molecule compound.

6. The method of claim 3, wherein the small molecule compound is selected from afatinib (Gilotrif), lapatinib (Tykerb), neratinib (Nerlynx), gefitinib (Iressa), erlotinib (Tarceva), vandetanib (Caprelsa), osimertinib (Tagrisso), dacomitinib (Vizimpro).

7-9. (canceled)

10. The method of claim 1, wherein the inhibitor is a peptide.

11. The method of claim 10, wherein the inhibitor is an antibody.

12. The method of claim 11, wherein the inhibitor is a monoclonal antibody (mAb).

13. The method of claim 12, wherein the monoclonal antibody is selected from Trastuzumab (Herceptin), cetuximab (Erbitux), panitumumab (Vectibix), necitumumab (Portrazza), pertuzumab (Omnitarg).

14. (canceled)

15. The method of claim 1, wherein the hematologic malignancy is selected from leukemia, non-Hodgkin lymphoma, Hodgkin lymphoma, multiple myeloma, and myelodysplastic syndromes.

16. The method of claim 15, wherein the hematologic malignancy is leukemia.

17-23. (canceled)

24. The method of claim 1, wherein the subject has prior treatment with another anti-cancer agent that is not an inhibitor of a member of the EGFR family.

25. The method of claim 24, wherein the subject has developed resistance to the other anti-cancer agent.

26. The method of claim 25, wherein the other anti-cancer agent is a Bruton's tyrosine kinase (BTK) inhibitor.

27-47. (canceled)

48. A method for affecting a micro environmental factor of a hematological cancer cell, comprising administering to a subject suffering from a hematologic malignancy, or a related disease or condition thereof, an inhibitor of a member of epidermal growth factor receptor (EGFR) family, or a pharmaceutically acceptable salt, ester or pro-drug thereof.

49. The method of claim 48, wherein the hematologic malignancy is selected from leukemia, non-Hodgkin lymphoma, Hodgkin lymphoma, multiple myeloma, and myelodysplastic syndromes.

50. The method of claim 49, wherein the hematologic malignancy is leukemia.
